(19) United States
(12) Patent Application Publication
(10) Pub. No.: US 2021/0350924 A1
(43) Pub. Date: Nov. 11, 2021

(54) Biological Information Analysis Apparatus, Biological Information Analysis Method, and Biological Information Analysis System

(71) Applicant: Nippon Telegraph and Telephone Corporation, Tokyo (JP)

(72) Inventors: Takayuki Ogasawara, Tokyo (JP); Kenichi Matsunaga, Tokyo (JP); Rieko Sato, Tokyo (JP)

(21) Appl. No.: 17/279,506

(22) PCT Filed: Sep. 11, 2019
(86) PCT No.: PCT/JP2019/035682

§ 371 Date: Mar. 24, 2021

(30) Foreign Application Priority Data
Sep. 26, 2018 (JP) 2018-179778
Feb. 28, 2019 (JP) 2019-035504

(51) Int. Cl. G16H 40/67; G01P 15/18; G16H 50/70; G16H 10/60; G16H 15/00; G06F 16/17

(52) U.S. Cl. CPC G16H 40/67; G01P 15/18; G06F 16/1734; G16H 10/60; G16H 15/00; G16H 50/70

(57) ABSTRACT

A biological information analysis apparatus includes a sensor data acquisition unit that acquires biological information of a user measured by a sensor, a storage unit that stores time-series data for the acquired biological information of the user, and an analysis unit that calculates statistical representative values of the time-series data for the biological information stored in the storage unit in a stepwise manner. The analysis unit includes a first calculation unit that calculates first representative values (intermediate representative values) from the time-series data for the biological information stored in the storage unit at intervals of a set period, and a second calculation unit that calculates a second representative value (a final representative value) from one intermediate representative value or multiple consecutive intermediate representative values.

### CROSS-REFERENCE TO RELATED APPLICATIONS

This application is a national phase entry of PCT Application No. PCT/JP2019/035682, filed on Sep. 11, 2019, which claims priority to Japanese Application No. 2018-179778, filed on Sep. 26, 2018, and Japanese Application No. 2019-035504, filed on Feb. 28, 2019, which applications are hereby incorporated herein by reference.

### TECHNICAL FIELD

The present invention relates to a biological information analysis apparatus, a biological information analysis method, and a biological information analysis system, and in particular to techniques for analyzing biological information which is measured by a sensor worn by a user.

### BACKGROUND

In recent years, biological information such as heart rate and acceleration measured by wearable devices and the like has been utilized in the fields of sports and medicine. For example, Non-Patent Literature 1 discloses a technique for estimating the posture or gait of a user based on measurement data from an acceleration sensor contained in a wearable device worn by the user.

In a biological information analysis system that uses a conventional sensor for measuring biological information of the user such as heart rate and acceleration, biological information is measured at a high-frequency sampling rate in order to capture biological activities thoroughly. Subsequently, analytic processing such as averaging can be performed on variations in the measured biological information caused by noise. Such analytic processing can reduce the effect of outliers contained in the measured biological information.

### CITATION LIST

### Non-Patent Literature

Non-Patent Literature 1: Kasai, Ogasawara, Nakashima, and Tsukada, “Development of Functional Textile “hitoe”: Wearable Electrodes for Monitoring Human Vital Signals”, The Institute of Electronics, Information and Communication Engineers, Communications Society Magazine No. 41 (June, 2017) (Vol. 11 No. 1).

### SUMMARY

### Technical Problem

Since biological information measured at high frequency entails a high data volume, it is desirable to reduce the number of data by thinning data to reduce the data to be transferred or to summarize biological information by averaging data at a sensor preceding transfer or calculating a proportion of measurement data occupied per certain time period in order to reduce the pieces of data, especially when biological information is transferred by wireless communication such as via Wi-Fi (a registered trademark).

However, averaging measured biological information and summarizing data in the conventional techniques requires storing measurement data for a certain period, so that reduction in data or summarization of biological information is difficult when a sensor with relatively low memory specifications is used.

Embodiments of the present invention have been made in order to solve this problem and an object thereof is to provide a biological information analysis apparatus, a biological information analysis method, and a biological information analysis system that enable data reduction and summarization of measured biological information even with a sensor having relatively low memory specifications.

### Means for Solving the Problem

To solve the problem, a biological information analysis apparatus according to embodiments of the present invention includes: a sensor data acquisition unit that acquires biological information of a user measured by a sensor; a storage unit that stores time-series data for the acquired biological information of the user; and an analysis unit that calculates statistical representative values of the time-series data for the biological information stored in the storage unit in a stepwise manner. The analysis unit includes: a first calculation unit that calculates first representative values from the time-series data for the biological information stored in the storage unit at intervals of a set period; and a second calculation unit that calculates a second representative value from one first representative value or a plurality of consecutive first representative values.

The biological information analysis apparatus according to embodiments of the present invention may further include an adjustment unit that determines the number of the first representative values to be used by the second calculation unit for calculating the second representative value, based on the number of the first representative values that are already calculated, and the second calculation unit may calculate the second representative value based on consecutive first representative values corresponding to the number of the first representative values determined by the adjustment unit.

The biological information analysis apparatus according to embodiments of the present invention may further include a determination unit that monitors for lack in the time-series data for the biological information and determines whether or not the lack is a long-term lack with a period of occurrence equal to or greater than a threshold at intervals of the set period, and the first calculation unit may not calculate the first representative values during the set period in which the lack determined to be a long-term lack by the determination unit is occurring.

The biological information analysis apparatus according to embodiments of the present invention may further include a second determination unit that determines that an anomaly has occurred when a value of the time-series data for the biological information falls in a set range of values for a certain period.

To solve the problem, a biological information analysis method according to embodiments of the present invention includes: a sensor data acquisition step of acquiring biological information of a user measured by a sensor; a storage step of storing time-series data for the acquired biological information of the user in a storage unit; and an analysis step of calculating statistical representative values of the time-series data for the biological information stored in the storage unit in a stepwise manner. The analysis step includes: a first calculation step of calculating first representative values from the time-series data for the biological information at intervals of a set period; and a second calculation step of calculating a second representative value from one first representative value or a plurality of consecutive first representative values.

To solve the problem, a biological information analysis system according to embodiments of the present invention includes: a sensor terminal that outputs biological information of a user measured by a sensor to outside; a relay terminal that receives the biological information of the user output by the sensor terminal and outputs the received biological information to outside; and an external terminal that receives the biological information of the user output by the sensor terminal or by the relay terminal and causes the received biological information to be displayed on a display device. At least one of the sensor terminal, the relay terminal, and the external terminal includes: a storage unit that stores time-series data for measured biological information; and an analysis unit that calculates statistical representative values of the time-series data for the biological information stored in the storage unit in a stepwise manner. The analysis unit includes: a first calculation unit that calculates first representative values from the time-series data for the biological information stored in the storage unit at intervals of a set period; and a second calculation unit that calculates a second representative value from one first representative value or a plurality of consecutive first representative values.

To solve the problem, a biological information analysis system according to embodiments of the present invention includes: a sensor terminal including a first analysis unit; a relay terminal including a second analysis unit; and an external terminal including a third analysis unit. The sensor terminal outputs biological information of a user measured by a sensor to outside, the relay terminal receives the biological information output by the sensor terminal and outputs the received biological information to outside, and the external terminal receives the biological information output by the sensor terminal or by the relay terminal and causes the received biological information to be displayed on a display device. At least one of the sensor terminal, the relay terminal, and the external terminal includes a storage unit that stores time-series data for the measured biological information. The first analysis unit, the second analysis unit and the third analysis unit cooperatively implement an analysis unit that calculates statistical representative values of the time-series data for the biological information stored in the storage unit in a stepwise manner. The analysis unit implements a first calculation unit that calculates first representative values from the time-series data for the biological information stored in the storage unit at intervals of a set period, and a second calculation unit that calculates a second representative value from one first representative value or a plurality of consecutive first representative values.

In the biological information analysis system according to embodiments of the present invention, the first analysis unit or the second analysis unit may constitute the first calculation unit, and the third analysis unit may constitute the second calculation unit.

In the biological information analysis system according to embodiments of the present invention, the relay terminal may be installed at a preset position and be capable of communication within a predetermined range from the position, and the relay terminal or the external terminal may include a third determination unit that determines that an anomaly has occurred when communication has been established between the relay terminal and the sensor terminal for a certain period.

In the biological information analysis system according to embodiments of the present invention, the first representative values and the second representative value may each be an average or a proportion of the biological information per certain period.

### Effects of Embodiments of the Invention

Embodiments of the present invention enable data reduction and summarization of measured biological information because they calculate statistical representative values of time-series data for biological information in a stepwise manner at intervals of a set period.

### BRIEF DESCRIPTION OF THE DRAWINGS

FIG. 1 is a block diagram describing functions of a biological information analysis apparatus according to a first embodiment of the present invention.

FIG. 2 is a block diagram showing a hardware configuration of the biological information analysis apparatus according to the first embodiment.

FIG. 3 is a flowchart illustrating a biological information analysis method according to the first embodiment.

FIG. 4 is a diagram describing calculation of representative values according to the first embodiment.

FIG. 5 shows a configuration of a biological information analysis system according to the first embodiment.

FIG. 6 is a block diagram showing a configuration of the biological information analysis system according to the first embodiment.

FIG. 7 is a sequence diagram illustrating operations of the biological information analysis system according to the first embodiment.

FIG. 8 is a block diagram describing functions of a biological information analysis apparatus according to a second embodiment.

FIG. 9 is a diagram describing an adjustment process according to the second embodiment.

FIG. 10 is a diagram describing a final representative value at a data end according to the second embodiment.

FIG. 11 is a sequence diagram illustrating operations of the biological information analysis system according to the second embodiment.

FIG. 12 is a diagram illustrating effects of the biological information analysis apparatus according to the second embodiment.

FIG. 13 is a block diagram describing functions of a biological information analysis apparatus according to a third embodiment.

FIG. 14 is a diagram describing a determination process according to the third embodiment.

FIG. 15 is a sequence diagram illustrating operations of the biological information analysis system according to the third embodiment.

FIG. 16 is a block diagram showing a configuration of a biological information analysis system according to a fourth embodiment.

FIG. 17 is a sequence diagram illustrating operations of a biological information analysis system according to a fifth embodiment.

FIG. 18 is a block diagram describing functions of a biological information analysis apparatus according to a sixth embodiment.

FIG. 19 is a flowchart illustrating the operations of the biological information analysis apparatus according to the sixth embodiment.

FIG. 20 is a block diagram illustrating functions of a biological information analysis apparatus according to a seventh embodiment.

FIG. 21 is a block diagram showing a configuration of the biological information analysis system according to the seventh embodiment.

### DETAILED DESCRIPTION OF ILLUSTRATIVE EMBODIMENTS

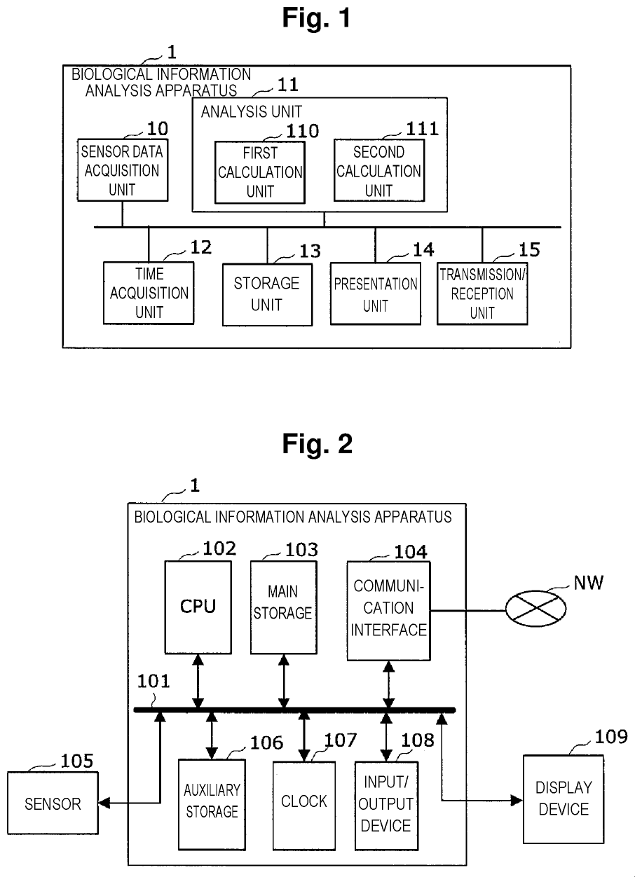

Preferred embodiments of the present invention are now described in detail with reference to FIGS. 1 through 21.

### First Embodiment

First, the configuration of a biological information analysis apparatus 1 according to a first embodiment of the present invention is generally described. FIG. 1 is a block diagram showing a functional configuration of the biological information analysis apparatus 1.

### Functional Blocks of Biological Information Analysis Apparatus

The biological information analysis apparatus 1 includes a sensor data acquisition unit 10, an analysis unit 11, a time acquisition unit 12, a storage unit 13, a presentation unit 14, and a transmission/reception unit 15.

The sensor data acquisition unit 10 acquires biological information of a user measured by a sensor 105 worn by the user, as discussed later. More specifically, when a heart rate meter is worn by the user as the sensor 105, for example, the sensor data acquisition unit 10 calculates a heart rate from electrocardiographic waveforms which are based on cardiac potentials measured by the heart rate meter. When an acceleration sensor is worn by the user as the sensor 105, the sensor data acquisition unit 10 converts an analog acceleration signal measured by the acceleration sensor to a digital signal at a predetermined sampling rate.

The sensor data acquisition unit 10 outputs time-series data in which the heart rate or the acceleration signal in the form of digital data and times of measurement are associated with each other. In this example, the heart rate and the acceleration data constitute biological information. The time-series data for the biological information measured by the sensor data acquisition unit 10 is stored in the storage unit 13, which is discussed later.

The analysis unit 11 includes a first calculation unit 110 and a second calculation unit 111. The analysis unit 11 calculates statistical representative values of the time-series data for the user's biological information stored in the storage unit 13. In this embodiment, the analysis unit 11 calculates representative values for biological information in two stages. The analysis unit 11 may calculate an average or a proportion of biological information per certain period, for example, as a representative value for biological information.

The first calculation unit 110 calculates a first representative value (hereinafter called an “intermediate representative value”), which is an intermediate representative value for biological information, at intervals of a set period from the time-series data for the user's biological information. The calculated intermediate representative values are accumulated in the storage unit 13. More specifically, the first calculation unit 110 calculates an intermediate representative value indicating an average in each 60 seconds, for example, with the time-series data for biological information acquired by the sensor data acquisition unit 10.

The second calculation unit 111 calculates a second representative value (hereinafter called a “final representative value”), which is the final representative value of the time-series data for biological information, based on one intermediate representative value or multiple consecutive intermediate representative values. The calculated final representative value is stored in the storage unit 13.

More specifically, the second calculation unit 111 calculates one final representative value using five consecutive intermediate representative values, for example. For example, based on five consecutive intermediate representative values that have been calculated at intervals of 60 seconds, the second calculation unit 111 calculates a representative value in a period of 5 minutes as the final representative value. The number of consecutive intermediate representative values that are used when the second calculation unit 111 calculates the final representative value can be set as desired.

The time acquisition unit 12 acquires a reference time to be used in the biological information analysis apparatus 1. The time acquisition unit 12 may acquire time information from a clock 107 included in the biological information analysis apparatus 1 or instead from a time server not illustrated, for example. The time information acquired by the time acquisition unit 12 is used in the sampling of biological information by the sensor data acquisition unit 10 or in the computation of a period in calculating intermediate representative values by the first calculation unit 110.

The storage unit 13 stores time-series data for the biological information of the user measured by the sensor data acquisition unit 10. The storage unit 13 also stores intermediate representative values calculated at intervals of a set period by the first calculation unit 110. The storage unit 13 also stores the final representative value calculated by the second calculation unit 111.

The presentation unit 14 presents the representative value calculated by the analysis unit 11. More particularly, the presentation unit 14 displays the final representative value for biological information on a display device 109, which is discussed later. The presentation unit 14 also generates and presents information for assisting the user based on the final representative value. The presentation unit 14 may output the information for assisting the user to the display device 109 or to an operation device (not shown), which is embodied by a sound output device, a light source, an actuator, a thermal instrument and the like.

The transmission/reception unit 15 receives sensor data indicating the biological information measured by the sensor 105, which is discussed later. The transmission/reception unit 15 can also send the final representative value for biological information provided by the analysis unit 11 to the outside over a communication network.

### Hardware Configuration of Biological Information Analysis Apparatus

Next, an exemplary hardware configuration of the biological information analysis apparatus 1 having the aforementioned functions is described with the block diagram of FIG. 2.

As shown in FIG. 2, the biological information analysis apparatus 1 can be implemented by a computer including a CPU 102, a main storage 103, a communication interface 104, an auxiliary storage 106, a clock 107, and an input/output device 108, which are interconnected via a bus 101, and a program for controlling these hardware resources, for example. The biological information analysis apparatus 1 is connected with the externally provided sensor 105 and the display device 109 each via the bus 101.

The main storage 103 pre-stores a program for the CPU 102 to perform various kinds of control and operations. The functions of the biological information analysis apparatus 1, including the analysis unit 11 shown in FIG. 1, are implemented by the CPU 102 and the main storage 103.

The communication interface 104 is an interface circuit for performing communication with various external electronic appliances over a communication network NW.

For the communication interface 104, an arithmetic interface and an antenna which support wireless data communication standards such as LTE, 3G, wireless LAN, and Bluetooth (a registered trademark) are used, for example. The transmission/reception unit 15 described in FIG. 1 is embodied by the communication interface 104.

The sensor 105 is embodied by a sensor, such as a heart rate meter, an electrocardiograph and an acceleration sensor, for example. The sensor 105 is worn by the user for a preset measurement period and measures biological information such as the heart rate, acceleration and the like of the user.

The auxiliary storage 106 is composed of a readable-writable storage medium and a drive for writing and reading various kinds of information such as programs and data to/from the storage medium. For the auxiliary storage 106, a hard disk or semiconductor memory such as flash memory can be used as storage media.

The auxiliary storage 106 has a storage area for storing time-series data for biological information measured by the sensor 105 and a program storage area for storing a program for the biological information analysis apparatus 1 to perform analysis processing on the biological information. The storage unit 13 described in FIG. 1 is embodied by the auxiliary storage 106. The auxiliary storage 106 may further have a backup area for backing up such data and programs, for example.

The clock 107 is composed of an internal clock built in the computer and the like and measures time. Time information obtained by the clock 107 is used in the sampling of biological information and calculation of representative values. Time information obtained by the clock 107 will be acquired by the time acquisition unit 12 described in FIG. 1.

The input/output device 108 is composed of an I/O terminal which receives input of signals from external appliances such as the sensor 105 and the display device 109, and outputs signals to external appliances.

The display device 109 functions as the presentation unit 14 of the biological information analysis apparatus 1. The display device 109 is embodied by a liquid crystal display and the like. The display device 109 also constitutes an operation device for outputting user assistance information, which is generated based on the final representative value for biological information.

### Biological Information Analysis Method

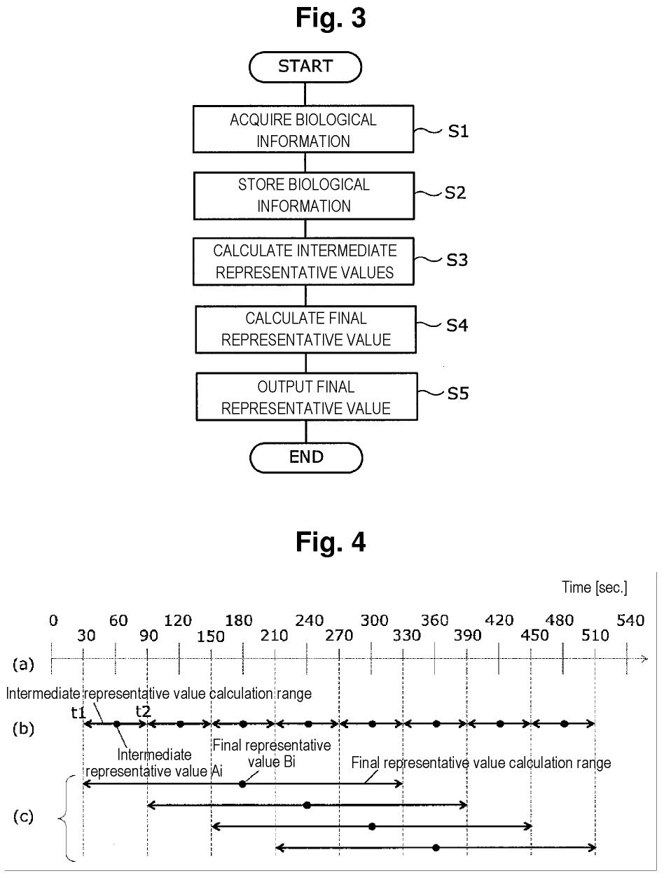

Next, the operation of the biological information analysis apparatus 1 having the above-described configuration is described with the flowchart of FIG. 3. First, the following processing is performed with the sensor 105 worn by the user.

The sensor data acquisition unit 10 acquires biological information measured by the sensor 105 worn by the user (step S1). More particularly, the sensor data acquisition unit 10 acquires the biological information and outputs time-series data in which the biological information and times of measurement are associated with each other. Next, the time-series data for the biological information is stored in the storage unit 13 (step S2).

Next, the first calculation unit 110 calculates intermediate representative values for the time-series data for the biological information measured at step S1 (step S3). More particularly, the first calculation unit 110 calculates an average of time-series data for biological information at intervals of a set period, e.g., at intervals of 60 seconds.

Subsequently, after a predetermined amount of time has passed, the second calculation unit 111 calculates the final representative value of the time-series data for biological information based on the intermediate representative values calculated at step S3 (step S4). More particularly, the second calculation unit 111 calculates a representative value for preset multiple consecutive intermediate representative values as the final representative value. For example, the second calculation unit 111 may calculate an average of five consecutive intermediate representative values after they are calculated at step S3. Then, the analysis unit 11 outputs the final representative value calculated at step S4 (step S5). The analysis unit 11 may also output the intermediate representative values calculated at step S3 together with the final representative value.

FIG. 4 is a diagram for describing an example of calculation of representative values by the analysis unit 11. The top row (a) shown in FIG. 4 shows a data sequence for biological information acquired by the sensor data acquisition unit 10 with elapsed time. The middle row (b) shows a sequence of intermediate representative values and measurement periods of biological data on which the respective intermediate representative values were calculated (hereinafter sometimes called a “calculation period”). The bottom row (c) shows a sequence of final representative values and the period of its calculation range.

Here, an intermediate representative value Aᵢ represents the i^{th} calculated intermediate representative value, with i being an integer equal to or greater than 1, and is represented in the form of a matrix. Given that the sum of measured values of biological information in a certain period is Sᵢ and the number of measured values is Nᵢ, the intermediate representative value Aᵢ represented by a vector is expressed by Formula (1):

$$\begin{matrix}Formula 1 & \\ {A}_{i} = ( {S}_{i} , {N}_{i} ) = ( {\sum }_{{t}_{1} = w ( i - 1 )}^{{t}_{2} = wi - 1} {a}_{t} , ( {t}_{2} - {t}_{1} ) / f ) & ( 1 )\end{matrix}$$

In Formula (1), aₜ is the measured value of biological information at measurement time t, and t₁ and t₂ are the starting and ending times of the period which is set for calculating intermediate representative values, respectively. Also, in Formula (1), the length of the calculation period is 60 seconds and biological information aₜ during it is sampled at a frequency of f [Hz]. Also, w is a constant defining a period included in measured values from which intermediate representative values are calculated. In this embodiment, w is 60 and the period slides by one calculation period, namely 60 seconds, every time i increases by 1 as an example.

Also, a final representative value Bᵢ is represented by Formula (2) below using Sᵢ and Nᵢ, which are components of the intermediate representative value Aᵢ represented by a vector. In Formula (2), S₋₁, S₀, N₋₁, N₀ are contained when i is 1 or 2, but these values are all set to 0.

$$\begin{matrix}Formula 2 & \\ {B}_{i} = \frac{{S}_{i - 2} + {S}_{i - 1} + {S}_{i} + {S}_{i + 1} + {S}_{i + 2}}{{N}_{i - 2} + {N}_{i - 1} + {N}_{i} + {N}_{i + 1} + {N}_{i + 2}} & ( 2 )\end{matrix}$$

Also, a stamped time T_{Bi} of the final representative value Bᵢ is represented by Formula (3) below. When the stamped time T_{Bi} is not an integer, it may be turned into an integer by rounding up the fractional part.

$$\begin{matrix}Formula 3 & \\ {T}_{{B}_{i}} = \frac{t 1 + t 2 + 1}{2} = \frac{w ( 2 i - 1 )}{2} & ( 3 )\end{matrix}$$

The functions of the biological information analysis apparatus 1 described above may also be distributed among multiple computers that are communicatively interconnected via a communication network, aside from being provided in a single computer.

### Biological Information Analysis System

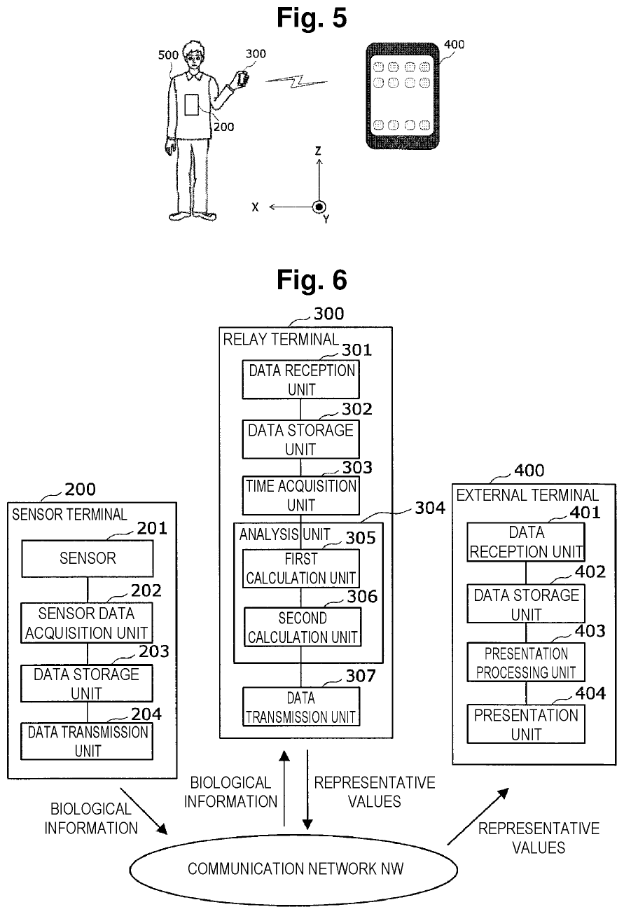

Next, a biological information analysis system as a specific arrangement of the biological information analysis apparatus 1 according to embodiments of the present invention is described with reference to FIGS. 5 and 6.

The biological information analysis system includes, for example, a sensor terminal 200 to be worn by a user 500, a relay terminal 300, and an external terminal 400 as shown in FIG. 5. All or some of the sensor terminal 200, the relay terminal 300, and the external terminal 400 have the functions of the biological information analysis apparatus 1, such as the analysis unit 11 described in FIG. 1. The following description is for a case where the relay terminal 300 includes the analysis unit 11 described in FIG. 1.

### Functional Blocks of Sensor Terminal

The sensor terminal 200 includes a sensor 201, a sensor data acquisition unit 202, a data storage unit 203, and a data transmission unit 204. The sensor terminal 200 is placed on a trunk of the user 500's body to measure biological information over multiple time intervals, for example. The sensor terminal 200 transmits the measured biological information of the user 500 to the relay terminal 300 over the communication network NW.

The sensor 201 is embodied by a heart rate meter, an acceleration sensor and the like. Three axes of the acceleration sensor included in the sensor 201 are set such that the X-axis is parallel to the right-left direction of the body, the Y-axis is to the front-back direction of the body, and the Z-axis is to the up-down direction of the body as shown in FIG. 5, for example. The sensor 201 corresponds to the sensor 105 described in FIG. 2.

The sensor data acquisition unit 202 acquires the biological information measured by the sensor 201. More particularly, the sensor data acquisition unit 202 performs removal of noise in the acquired biological information if necessary, performs sampling processing, and determines time-series data for biological information in the form of a digital signal. The sensor data acquisition unit 202 corresponds to the sensor data acquisition unit 10 described in FIG. 1.

The data storage unit 203 stores the biological information detected by the sensor 201 and time-series data for biological information in the form of a digital signal resulting from processing by the sensor data acquisition unit 202. The data storage unit 203 corresponds to the storage unit 13 (FIG. 1).

The data transmission unit 204 transmits the time-series data for the biological information stored in the data storage unit 203 to the relay terminal 300 over the communication network NW. The data transmission unit 204 includes a communication circuit for performing wireless communication which supports wireless data communication standards such as LTE, 3G, wireless LAN (Local Area Network), and Bluetooth (a registered trademark), for example. The data transmission unit 204 corresponds to the transmission/reception unit 15 (FIG. 1).

### Functional Blocks of Relay Terminal

The relay terminal 300 includes a data reception unit 301, a data storage unit 302, a time acquisition unit 303, an analysis unit 304, and a data transmission unit 307. The relay terminal 300 determines statistical representative values in a stepwise manner from the time-series data for the biological information of the user 500 received from the sensor terminal 200. Further, the relay terminal 300 transmits the calculated representative values to the external terminal 400.

The relay terminal 300 is embodied by a smartphone, a tablet, a notebook PC and the like.

The data reception unit 301 receives time-series data for biological information from the sensor terminal 200 over the communication network NW. The data reception unit 301 corresponds to the transmission/reception unit 15 (FIG. 1).

The data storage unit 302 stores the biological information of the user 500 received by the data reception unit 301 and the representative values for biological information provided by the analysis unit 304. The data storage unit 302 corresponds to the storage unit 13 (FIG. 1).

The time acquisition unit 303 acquires time information to be used in analysis processing for the biological information by the analysis unit 304 from the internal clock (the clock 107). The time acquisition unit 303 corresponds to the time acquisition unit 12 described in FIG. 1.

The analysis unit 304 includes a first calculation unit 305 and a second calculation unit 306.

The analysis unit 304 determines, in a stepwise manner, statistical representative values, such as averages, of the time-series data for the biological information of the user 500 received by the data reception unit 301. The analysis unit 304 corresponds to the analysis unit 11 described in FIG. 1.

The first calculation unit 305 calculates an intermediate representative value from time-series data for the biological information of the user 500 at intervals of a set period, e.g., at intervals of 60 seconds. The calculated intermediate representative value is stored in the data storage unit 302.

The second calculation unit 306 calculates the final representative value from multiple consecutive intermediate representative values.

The first calculation unit 305 and the second calculation unit 306 correspond to the first calculation unit 110 and the second calculation unit 111 described in FIG. 1, respectively.

The data transmission unit 307 transmits the final representative value calculated by the second calculation unit 306 to the external terminal 400 over the communication network NW. The data transmission unit 307 corresponds to the transmission/reception unit 15 (FIG. 1). The data transmission unit 307 may also transmit intermediate representative values with the final representative value.

### Functional Blocks of External Terminal

The external terminal 400 includes a data reception unit 401, a data storage unit 402, a presentation processing unit 403, and a presentation unit 404. The external terminal 400 presents the final representative value for the biological information of the user 500 received from the relay terminal 300 over the communication network NW and also presents assistance information for the user 500 which is generated based on the calculated final representative value.

The external terminal 400 is embodied by a smartphone, a tablet, a notebook PC and the like as with the relay terminal 300. The external terminal 400 has a display device for displaying the received final representative value and an operation device (not shown) for outputting information for assisting the user 500 which is generated based on the calculated final representative value. Examples of the operation device provided in the external terminal 400 include a display device, a sound output device, a light source, an actuator, and a thermal instrument.

The sound output device can be a speaker or a musical instrument, for example. The light source can be an LED or a light bulb. The actuator can be a vibrator, a robot arm, or an electric therapy machine. The thermal instrument can be a heater, a Peltier device and the like.

The data reception unit 401 receives the final representative value for biological information from the relay terminal 300 over the communication network NW. The data reception unit 401 corresponds to the transmission/reception unit 15 (FIG. 1).

The data storage unit 402 stores the final representative value for biological information received by the data reception unit 401. The data storage unit 402 corresponds to the storage unit 13 (FIG. 1).

The presentation processing unit 403 generates assistance information for the user 500 based on the received final representative value. The presentation processing unit 403 corresponds to the presentation unit 14 described in FIG. 1.

The presentation unit 404 presents the final representative value and assistance information for the user 500 according to an instruction from the presentation processing unit 403. More particularly, the presentation unit 404 may display the final representative value and assistance information on the display device of the external terminal 400 in the form of textual information, a graph and the like, or output assistance information via an alert sound from a speaker, not shown, provided in the external terminal 400. In addition, the presentation unit 404 can present the assistance information in a manner perceivable by the user 500, such as via vibration and light. The presentation unit 404 corresponds to the presentation unit 14 described in FIG. 1.

As shown above, the biological information analysis system according to embodiments of the present invention is configured such that the functions of the biological information analysis apparatus 1 are distributed across the sensor terminal 200, the relay terminal 300 and the external terminal 400, and performs processing related to measurement of biological information of the user 500, calculation of representative values, and presentation of the final representative value in a distributed manner.

### Operational Sequence of Biological Information Analysis System

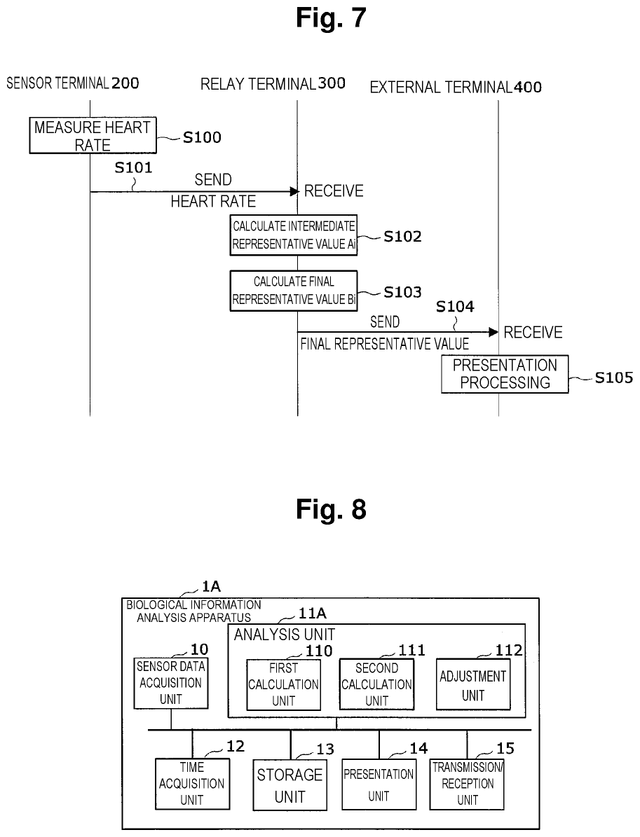

Next, the operation of the biological information analysis system having the above-described configuration is described with the sequence diagram of FIG. 7. In the following, representative values for the heart rate of the user 500 are determined as an example of biological information.

As shown in FIG. 7, initially, the sensor terminal 200 worn by the user 500 measures the heart rate of the user 500 (step S100). More particularly, the sensor 201 composed of a heart rate meter measures the cardiac potentials of the user 500. The sensor data acquisition unit 202 acquires the heart rate of the user 500 from electrocardiographic waveforms which are based on the cardiac potentials.

Next, the sensor terminal 200 transmits time-series data for the heart rate of the user 500 to the relay terminal 300 over communication network NW (step S101). After receiving the time-series data for the heart rate from the sensor terminal 200, the relay terminal 300 calculates an intermediate representative value at intervals of the set period (step S102). More particularly, the first calculation unit 305 calculates the intermediate representative value Aᵢ at intervals of 60 seconds using the Formula (1) mentioned above.

Next, after elapse of a predetermined amount of time, the second calculation unit 306 calculates the final representative value from multiple consecutive intermediate representative values (step S103). More particularly, the second calculation unit 306 uses the Formula (2) mentioned above to calculate the final representative value Bᵢ with five consecutive intermediate representative values. The second calculation unit 306 can determine the stamped time T_{Bi} of the final representative value Bᵢ according to the Formula (3) mentioned above.

In this manner, by using intermediate representative values in determining representative values of time-series data for the acquired biological information, an original size of 300 pieces of data, for example, can be reduced to as low as 10 intermediate representative values as the element of a matrix and then the final representative value can be calculated. Also, since it is possible to repeat calculation of the final representative value Bᵢ in a stage preceding data transfer, an effect equivalent to applying a moving average and then down-sampling data at the interval of certain periods (at intervals of 60 seconds in this embodiment) can be obtained. It thus provides the effect of reducing the data volume to be transferred.

Referring back to FIG. 7, the relay terminal 300 subsequently transmits the final representative value of the time-series data for the user 500's heart rate to the external terminal 400 over the communication network NW (step S104). After receiving the final representative value, the external terminal 400 performs presentation processing (step S105). That is, the external terminal 400 causes the final representative value to be displayed on the display device. The external terminal 400 also generates assistance information for the user 500 based on the final representative value and causes it to be displayed on the display device and the like.

As described above, the biological information analysis apparatus 1 according to the first embodiment calculates intermediate representative values from time-series data for biological information at intervals of a set period, and calculates the final representative value based on multiple consecutive intermediate representative values. Accordingly, the biological information analysis apparatus 1 can enable data reduction and summarization for measured biological information concurrently.

### Second Embodiment

Next, a second embodiment of the present invention is described. In the following descriptions, the same components as those in the first embodiment above are denoted with the same reference numerals and description thereof is omitted.

The first embodiment showed a case where the second calculation unit 111 calculates the final representative value based on a preset number of intermediate representative values. By contrast, in the second embodiment, an analysis unit 11A further includes an adjustment unit 112. The adjustment unit 112 enables varying of the number of intermediate representative values to be used by the second calculation unit 111 for calculating the final representative value based on the number of intermediate representative values that can be secured in the past or future. The second embodiment is described below focusing on different arrangements from the first embodiment.

As shown in FIG. 8, the biological information analysis apparatus 1A includes the analysis unit 11A. The analysis unit 11A includes a first calculation unit 110, the second calculation unit 111, and the adjustment unit 112. The other functional components of the biological information analysis apparatus 1A are similar to the first embodiment.

The adjustment unit 112 determines the number of intermediate representative values to be used by the second calculation unit 111 for calculating the final representative value based on the number of intermediate representative values that are already calculated. More particularly, the adjustment unit 112 monitors whether there are a sufficient number of intermediate representative values for calculating the final representative value each time the second calculation unit 111 calculates the final representative value. For example, the adjustment unit 112 counts five consecutive intermediate representative values, which are necessary when the second calculation unit 111 calculates the final representative value Bᵢ using the previously mentioned Formula (2).

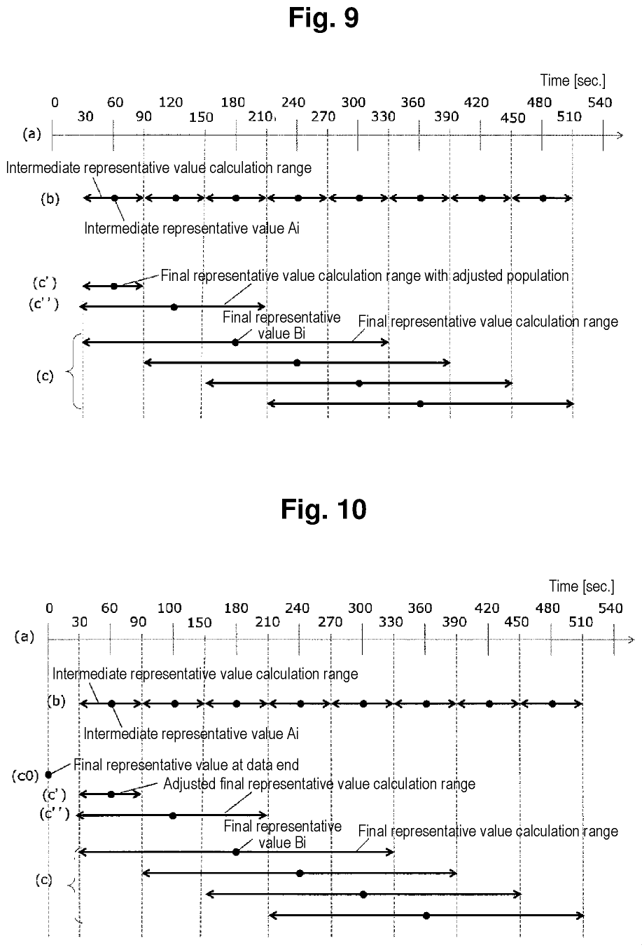

More specifically, as shown in FIG. 9, five consecutive intermediate representative values Aᵢ are necessary for the second calculation unit 111 to calculate the final representative value Bᵢ shown in the bottom row (c). If the second calculation unit 111 calculates the final representative value Bᵢ in response to settings or an external signal at 120 seconds after the start of measurement of biological information, for example, only one intermediate representative value Aᵢ has been calculated as opposed to the required number of five intermediate representative values Aᵢ.

In this case, the adjustment unit 112 adjusts the required number of intermediate representative values Aᵢ for the second calculation unit 111 to calculate the final representative value. Then, the adjustment unit 112 adopts the lesser one of either the number of intermediate representative values Aᵢ that are already calculated at the point the second calculation unit 111 calculates the final representative value or the number of intermediate representative values Aᵢ that will be calculated after that point.

That is, if the second calculation unit 111 calculates the final representative value Bᵢ at 120 seconds after the start of measurement of biological information, only one intermediate representative value Aᵢ has been calculated; thus, only one value is used also for the intermediate representative value Aᵢ that will be calculated in the future, and the final representative value Bᵢ is calculated based on a total of three intermediate representative values Aᵢ (the bottom row (c″) in FIG. 9).

Giving another example, if the second calculation unit 111 calculates the final representative value Bᵢ 60 seconds after the start of measurement of biological information, for example, zero intermediate representative values Aᵢ have been calculated at 60 seconds. Thus, the adjustment unit 112 adopts 0 also for the number of intermediate representative values Aᵢ that will be calculated in the future. In this case, the second calculation unit 111 handles the intermediate representative value Aᵢ directly as the final representative value Bᵢ (the bottom row (c′) in FIG. 9).

As shown in FIG. 10, at a data end, a measured value of biological information itself may be complementarily used as the final representative value (the bottom row (co) in FIG. 10). With such adjustment or complementing, the final representative value Bᵢ can be calculated even in cases where i is 1 or 2, which prevents determination of the final representative value Bᵢ by the Formula (2) mentioned above.

### Operational Sequence of Biological Information Analysis System

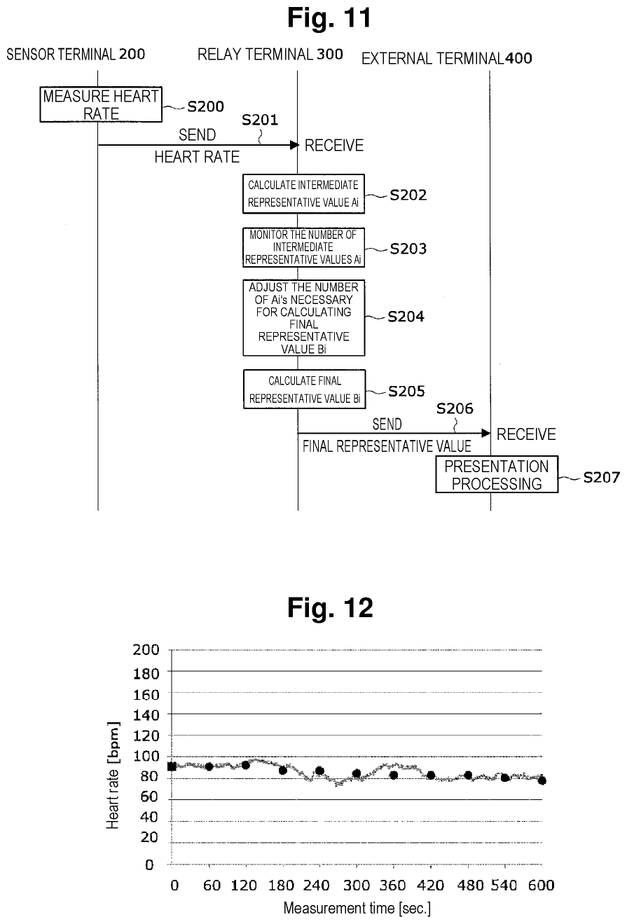

Next, operations in a case where the functions of the biological information analysis apparatus 1A according to this embodiment are implemented by a biological information analysis system including the sensor terminal 200, the relay terminal 300 and the external terminal 400 described in FIG. 6 are described with reference to the sequence diagram shown in FIG. 11. The respective functional blocks of the sensor terminal 200, the relay terminal 300, and the external terminal 400 are similar to the configurations described in FIG. 6. Also, it is assumed that the relay terminal 300 includes the adjustment unit 112.

Initially, the sensor terminal 200 is attached to the user 500 and measures the heart rate, for example, as the biological information of the user 500 (step S200). More specifically, the sensor terminal 200 detects the cardiac potentials of the user 500 with the heart rate meter (the sensor 201). The sensor data acquisition unit 202 acquires cardiac potentials from the sensor 201 and calculates the heart rate from electrocardiographic waveforms which are based on the cardiac potentials. The acquired cardiac potentials and the heart rate are stored in the data storage unit 203.

Next, the sensor terminal 200 transmits the measured heart rate to the relay terminal 300 over the communication network NW (step S201). More specifically, the data transmission unit 204 reads time-series data for the heart rate from the data storage unit 203 and transmits it to the relay terminal 300 over the communication network NW.

After the relay terminal 300 receives the time-series data for the heart rate of the user 500 from the sensor terminal 200, the first calculation unit 305 calculates intermediate representative values of the time-series data for the heart rate at intervals of a set period, e.g., at intervals of 60 seconds (step S202). The calculated intermediate representative values are stored in the data storage unit 302.

Next, the adjustment unit 112 monitors the number of intermediate representative values calculated by the first calculation unit 305 (step S203). Subsequently, in response to, for example, an external signal or settings, at the point when the second calculation unit 306 calculates the final representative value, the adjustment unit 112 determines the number of intermediate representative values necessary for the calculation of the final representative value based on the number of already calculated intermediate representative values (step S204).

Then, based on consecutive intermediate representative values corresponding to the number of intermediate representative values determined by the adjustment unit 112, the second calculation unit 306 calculates the final representative value (step S205). Next, the calculated final representative value is transmitted from the relay terminal 300 to the external terminal 400 (step S206).

Subsequently, the external terminal 400 receives the final representative value. The external terminal 400 performs presentation processing based on the final representative value (step S207), displaying the final representative value on the display device or generating and outputting assistance information for the user 500.

### Effects of the Second Embodiment

Next, referring to FIG. 12, the effects of the biological information analysis apparatus 1A according to this embodiment are described. In FIG. 12, the horizontal axis indicates measurement time (seconds) and the vertical axis indicates the heart rate (bpm). The gray line shown in FIG. 12 indicates the measured heart rate and circle and square dots indicate final representative values. The final representative value at the measurement time of 0 seconds uses the measured heart rate value as it is (the square dot).

The two circle dots indicating the final representative values of the heart rate at the subsequent measurement times 60 seconds and 120 seconds indicate final representative values that were calculated with the number of intermediate representative values determined by the adjustment unit 112. Further, the circle dots at and after the measurement time of 180 seconds indicate final representative values that were calculated with five intermediate representative values without adjustment to the number of intermediate representative values by the adjustment unit 112.

As shown in FIG. 12, while the measured value of the heart rate (the gray line) varies up and down, it can be seen that the final representative value, which transitions in almost the center of the measured value, is appropriate as a down-sampled moving average.

As described above, according to the biological information analysis apparatus 1A in the second embodiment, the adjustment unit 112 determines the number of intermediate representative values that are used when the second calculation unit 111 calculates the final representative value based on the number of intermediate representative values that are already calculated. Thus, data around the start or the end of measurement of biological information can be effectively utilized and the behavior of the user's biological information can be ascertained more precisely.

Although the second embodiment above showed a case where the analysis unit 11A includes the adjustment unit 112, the adjustment unit 112 may be provided outside the analysis unit 11A in the biological information analysis apparatus 1A.

### Third Embodiment

Next, a third embodiment of the present invention is described. In the following descriptions, the same components as those in the first and second embodiments above are denoted with the same reference numerals and description thereof is omitted.

In the first and second embodiments, representative values of time-series data for biological information are determined assuming that the biological information measured by the sensor data acquisition unit 10 has no lack. By contrast, in the third embodiment, an analysis unit 11B further includes a first determination unit 113. The first determination unit 113 determines calculation processing for intermediate representative values by the first calculation unit 110 in accordance with the situation of lack in the biological information. The third embodiment is described below focusing on different arrangements from the first and second embodiments.

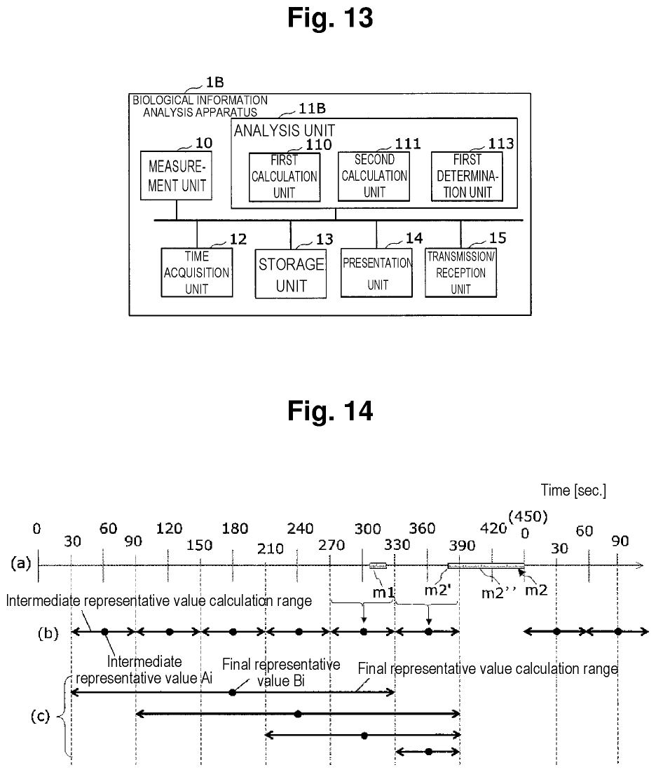

As shown in FIG. 13, the analysis unit 11B of a biological information analysis apparatus 1B includes the first calculation unit 110, the second calculation unit 111, and the first determination unit 113. The other components of biological information analysis apparatus 1B are similar to the first embodiment.

The first determination unit 113 monitors for lack in time-series data for biological information and determines whether the lack is a long-term lack with a period of occurrence equal to or greater than a threshold, at intervals of a set period, e.g., at intervals of 60 seconds. More particularly, the first determination unit 113 uses a period value of “20 seconds” for the threshold, for example, and determines that a lack occurring in the time-series data for biological information is a short-term lack if its period of occurrence is less than 20 seconds. On the other hand, when the period of a lack occurring in the time-series data for biological information is equal to or greater than 20 seconds, the first determination unit 113 determines that the lack is a long-term lack.

The first calculation unit 110 calculates an intermediate representative value at intervals of the set period based on the result of determination by the first determination unit 113. More particularly, when the first determination unit 113 determines that a lack occurring in the time-series for biological information is a long-term lack in a set period of 60 seconds, the first calculation unit 110 does not calculate an intermediate representative value in that 60-second period. On the other hand, when the first determination unit 113 determines that a lack occurring in the time-series for biological information is a short-term lack in the set period of 60 seconds, the first calculation unit 110 calculates an intermediate representative value excluding that lack of data.

Now referring to FIG. 14, a specific example of a determination process by the first determination unit 113 is described. In the measurement data sequence for biological information shown in the top row (a) of FIG. 14, there is a lack m1 occurring in a period set for calculation of an intermediate representative value, from 270 seconds to 330 seconds. Also, there is a lack m2 occurring across two periods, the period from 330 seconds to 390 seconds and the period from 390 seconds to around 450 seconds. More particularly, a portion m2′ of the lack m2 is included in the period from 330 seconds to 390 seconds and another portion m2″ of the lack m2 is occurring in the period from 390 seconds to around 450 seconds.

For the example of FIG. 14, it is assumed that the periods of the lacks m1, m2′ are less than 20 seconds and the period of the lack m2″ is equal to or greater than 20 seconds. First, the first determination unit 113 determines that the lack m1 occurring in the period from 270 seconds to 330 seconds is a short-term lack as the period of the lack m1 is less than the threshold (20 seconds). The first calculation unit 110 calculates an intermediate representative value for biological information in the period from 270 seconds to 330 seconds based on the result of determination that the lack m1 is a short-term lack.

For example, assume that the period of the lack m1 is 10 seconds where the sampling rate is one second in measurement of biological information. In this case, the number Nᵢ of measured values of biological information constituting the intermediate representative value Aᵢ in the Formula (1) mentioned above is normally Nᵢ=60, but Nᵢ is set to Nᵢ=50 due to the occurrence of the lack m1. Similarly, for the period from 330 seconds to 390 seconds, the lack m2′ is a short-term lack less than 20 seconds, so that the first calculation unit 110 calculates the intermediate representative value Aᵢ with the previously mentioned Formula (1) excluding the lack m2′.

As to the lack m2″ occurring in the period from 390 seconds to around 450 seconds, the first determination unit 113 determines that the lack m2″ is a long-term lack because it is equal to or greater than the threshold (20 seconds). In this case, the first calculation unit 110 does not calculate an intermediate representative value for the set 60-second period from 390 seconds to 450 seconds.

As shown in the example of FIG. 14, intermediate representative values are calculated at intervals of the set 60-second period from a new starting point, which is the data end at around 450 seconds, at which the long-term lack m2 (m2″) occurring in the measurement data sequence shown in the top row (a) ends.

As another example of a determination process by the first determination unit 113, the first determination unit 113 may monitor for a lack in the data sequence for biological information, determine whether the lack is a short-term lack or a long-term lack by comparison to the threshold, and only if the lack is determined to be a long-term lack, it may further determine whether the long-term lack is a short-term lack or a long-term lack within the set 60-second period for calculating an intermediate representative value.

### Operational Sequence of Biological Information Analysis System

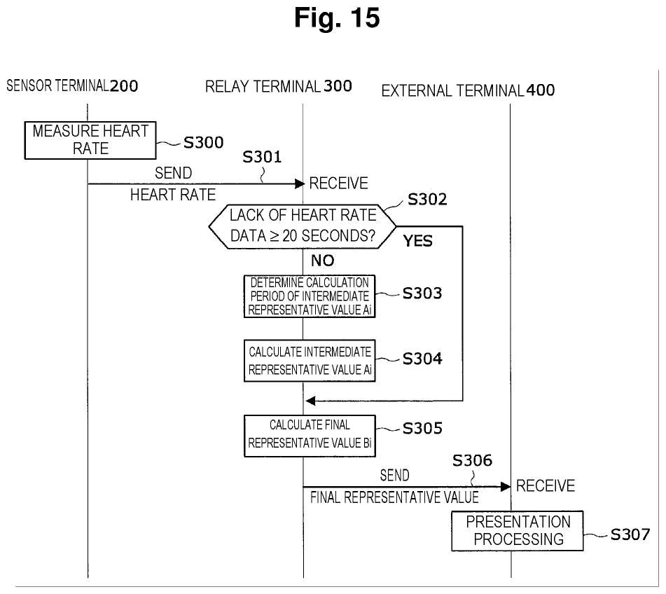

Next, the operation is described with the sequence diagram of FIG. 15 taking as an example a case where the functions of the biological information analysis apparatus 1B according to this embodiment are implemented by a biological information analysis system including the sensor terminal 200, the relay terminal 300 and the external terminal 400 described in FIG. 6. This embodiment shows a case where the relay terminal 300 includes the analysis unit 11B having the first determination unit 113.

Initially, the sensor terminal 200 is attached to the user 500 and measures the heart rate, for example, as the biological information of the user 500 (step S300). More specifically, the sensor terminal 200 detects the cardiac potentials of the user 500 with the heart rate meter (the sensor 201). The sensor data acquisition unit 202 acquires cardiac potentials from the sensor 201 and calculates the heart rate from electrocardiographic waveforms which are based on the cardiac potentials. The acquired cardiac potentials and the heart rate are stored in the data storage unit 203.

Next, the sensor terminal 200 transmits the measured heart rate to the relay terminal 300 over the communication network NW (step S301). More specifically, the data transmission unit 204 reads time-series data for the heart rate from the data storage unit 203 and transmits it to the relay terminal 300 over the communication network NW.

After the relay terminal 300 receives the time-series data for the heart rate of the user 500 from the sensor terminal 200, the first determination unit 113 monitors for lack in the received time-series data for the heart rate. Then, if the period of a lack occurring in the 60-second period during which an intermediate representative value is calculated is less than 20 seconds (step S302: NO), the determination unit 113 determines that it is a short-term lack.

Subsequently, the first calculation unit 305 determines the period for calculating intermediate representative values based on the result of determination (step S303). Next, the first calculation unit 305 calculates an intermediate representative value using measurement data for biological information excluding the lack and using the period determined at step S303 (step S304). More particularly, the first calculation unit 305 utilizes the Formula (1) above to calculate an intermediate representative value.

On the other hand, when the period of the lack occurring in the time-series data for the heart rate is equal to or greater than 20 seconds in the 60-second period during which an intermediate representative value is calculated (step S302: YES), the first determination unit 113 determines that it is a long-term lack. In this case, the first calculation unit 305 does not calculate an intermediate representative value in that set 60-second period based on the result of the determination.

Subsequently, the second calculation unit 306 calculates the final representative value using the Formula (2) above and based on five consecutive intermediate representative values (step S305). The second calculation unit 306 can also determine the stamped time of the final representative value with the Formula (3) above. If there is a long-term lack occurring in a 60-second period of the time-series data for the heart rate measured by the first determination unit 113, the final representative value is calculated with the starting point of the 60-second period in which the long-term lack is included (at 390 seconds in FIG. 14) as the end of data.

Further, the adjustment unit 112 used in the second embodiment may be employed to adjust the number of intermediate representative values for use in the calculation of the final representative value with respect to the calculation of the final representative value near an end of data (the bottom row (c) of FIG. 14).

Referring back to FIG. 15, the relay terminal 300 transmits the final representative value to the external terminal 400 over the communication network NW (step S306).

Subsequently, the external terminal 400 receives the final representative value. The external terminal 400 performs presentation processing based on the final representative value (step S307), displaying the final representative value on the display device or generating and outputting assistance information for the user 500.

As described above, according to the biological information analysis apparatus 1B of the third embodiment, when the first determination unit 113 determines that a lack of biological information occurring in a set period is a long-term lack, the first calculation unit 110 does not calculate the intermediate representative value for that set period. When the lack is a short-term lack, it calculates an intermediate representative value excluding the lack. Thus, calculation of an abnormal representative value due to lack can be prevented and representative values for biological information that are truly reliable can be presented.

The above embodiment shows the example of calculating intermediate representative values in consideration of any lack in the measured biological information. However, the determination unit 113 may also monitor for the occurrence of outliers in biological information in addition to lack and apply a determination process in a similar manner. Excluding data for biological information which is determined to be an outlier by the first determination unit 113, the rest is similar to the case of lack. For example, the determination unit 113 can employ thresholding or identification with machine learning for determination of outliers and determine an outlier with respect to the heart rate or blood pressure of a healthy subject (e.g., the value of heart rate or blood pressure being 0 and the like).

Also, while the described embodiment described a case where the analysis unit 11B includes the first determination unit 113, the determination unit 113 may be provided outside the analysis unit 11B in the biological information analysis apparatus 1B.

### Fourth Embodiment

Next, a fourth embodiment of the present invention is described. In the following descriptions, the same components as those in the first to third embodiments are denoted with the same reference numerals and description thereof is omitted.

The first to third embodiments showed a case where the relay terminal 300 includes the analysis unit 11 and calculates intermediate representative values and a final representative value in the biological information analysis system. By contrast, the biological information analysis system according to the fourth embodiment performs calculation of intermediate representative values and calculation of the final representative value on different terminals in a distributed manner. The fourth embodiment is described below focusing on different arrangements from the first to third embodiments.

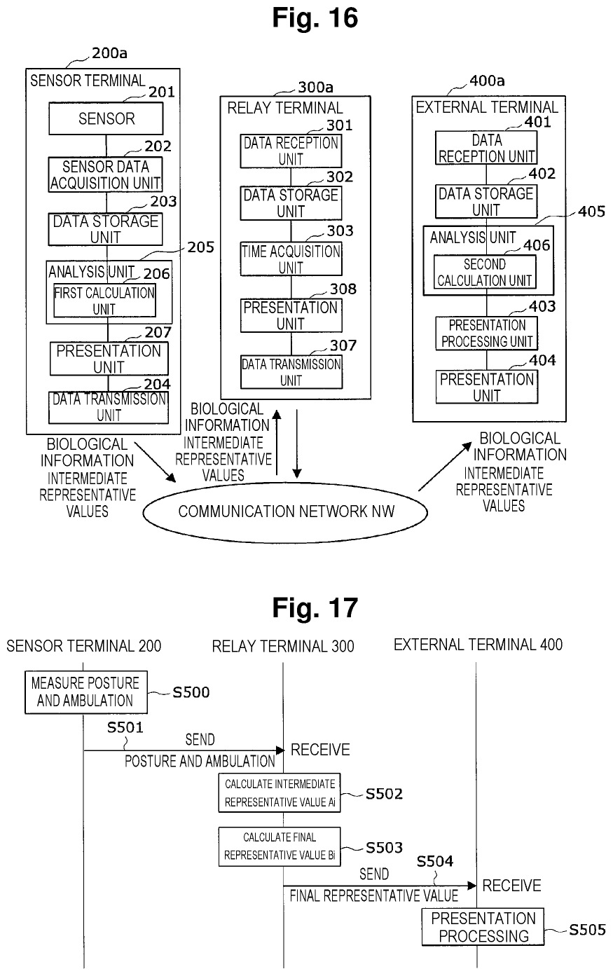

As shown in FIG. 16, the biological information analysis system according to the fourth embodiment includes a sensor terminal 200a, a relay terminal 300a, and an external terminal 400a, which are communicatively connected with each other via the communication network NW.

The sensor terminal 200a includes the sensor 201, the sensor data acquisition unit 202, the data storage unit 203, the analysis unit 205, the presentation unit 207, and the data transmission unit 204. The analysis unit 205 has a first calculation unit 206. The first calculation unit 206 calculates intermediate representative values of time-series data for biological information at intervals of a set period. The calculated intermediate representative values are stored in the data storage unit 203. The presentation unit 207 also causes the calculated intermediate representative values to be displayed on the display device and the like.

The relay terminal 300a includes a data reception unit 301, a data storage unit 302, a time acquisition unit 303, a presentation unit 308, and a data transmission unit 307. The presentation unit 308 causes the intermediate representative values received from the sensor terminal 200a to be displayed on the display device and the like.

The external terminal 400a includes a data reception unit 401, a data storage unit 402, an analysis unit 405, a presentation processing unit 403, and a presentation unit 404. The analysis unit 405 has a second calculation unit 406. The external terminal 400a receives intermediate representative values from the relay terminal 300a over the communication network NW.

The second calculation unit 406 calculates the final representative value based on multiple consecutive intermediate representative values calculated by the first calculation unit 206 of the sensor terminal 200a. The presentation unit 404 causes the calculated final representative value to be displayed on the display device and the like.

By thus determining and displaying intermediate representative values for biological information, which are calculated more frequently than the calculation of the final representative value, at the sensor terminal 200a measuring biological information, a demand for viewing biological information in a more real-time manner can be addressed. The calculation of the final representative value in particular requires memory of a larger capacity because consecutive, e.g., five, intermediate representative values are used. Calculation of intermediate representative values only, in contrast, can be implemented even by a sensor terminal 200a having relatively low memory specifications required for data retention.

In the case where a sensor terminal 200a with lower memory specifications is used and implementation of analysis and viewing functions on the sensor terminal 200a is difficult, calculation and display of intermediate representative values may be performed at the relay terminal 300a.

Also, when intermediate representative values and the final representative value are calculated on different terminals as in this embodiment, securing intermediate representative values as real-time information, rather than handling them only as intermediate products for the calculation of the final representative value, enables the user behavior to be checked promptly with less delay such as during the measurement of biological information.

### Fifth Embodiment

Next, a fifth embodiment of the present invention is described. In the following descriptions, the same components as those in the first to fourth embodiments are denoted with the same reference numerals and description thereof is omitted.

The first to fourth embodiments illustrated the case of calculating averages as intermediate representative values and final representative values of time-series data for biological information. By contrast, the fifth embodiment determines proportions, not an average, as a statistical representative value of time-series data for biological information.

The configuration of the biological information analysis apparatus 1 according to this embodiment is similar to that of the biological information analysis apparatus 1 shown in FIG. 1. The configuration of the biological information analysis system according to this embodiment is also similar to the configuration shown in FIG. 6. The fifth embodiment is described below focusing on different arrangements from the first to fourth embodiments.

Heart rate, body temperature and blood pressure as a kind of biological information permit calculation of an average in time order as shown in the first embodiment. However, for other kinds of biological information, it is not necessarily appropriate to perform such average calculation. For example, this can be the case when the state of the user is estimated with a sensor. Where a state of the user being couched (lying) is defined as 0 and a state of the user being upright is defined as 1 and the user's state is always classified into either of the two values, a value of 0.5, namely the average of the values, is not meaningful.

Information that can assume an intermediate value, such as heart rate, is referred to as a quantitative variable, and information that does not permit an intermediate value representing a state, such as posture, is referred to as a qualitative variable. When the biological information analysis apparatus 1 analyzes such a qualitative variable, it is desirable to use proportions representing the frequency of occurrence thereof.

For example, assume that a couched (lying) state is defined as 0, an upright state is defined as 1, and a walking state is defined as 2, and an intermediate representative value A_{j,i} is determined at intervals of 60 seconds with a sampling rate of 1 second. A_{j,i} is the i^{th} intermediate representative value, assuming a state of j. In this case, j is any one of 0, 1, 2. Then, if a couched period is included for 30 seconds, an upright period is included for 20 seconds, and a walking period is included for 10 seconds, it is represented by Formula (4) below with the those numbers being N_{0,i}, N_{1,i}, N_{2,i}, respectively.

Formula 4

A_{j,i}=j when N_{j,i}=MAX(N_{0,i},N_{1,i},N_{2,i}) (4)

Then, MAX(N_{0,i}, N_{1,i}, N_{2,i})=MAX(30, 20, 10)=30 after “when” in Formula (4) gives j=0, that is, A_{0,i}=0, so that a couched state is selected as a mode. Also, to calculate the final representative value Bᵢ for the user's state with the mode of the intermediate representative value A_{j,i}, the final representative value Bᵢ is calculated with Formula (5) below.

Formula 5

Bᵢ=MODE(A_{j,i-2},A_{j,i-1},A_{j,i},A_{j,i+1},A_{j,i+2}) (5)

Here, Formula (5) above is for the case of including five intermediate representative values A_{j,i}. MODE indicates an operation to select the most frequent one of the components in an associated vector, where Bᵢ=MODE (1, 2, 0, 1, 1)=1. A condition such as giving priority to one with a greater value if multiple candidates for the mode have arisen may be defined in advance. However, the arrangement of adopting the mode of the user's state as the intermediate representative value A_{j,i} or the final representative value Bᵢ is an example and other ways of determination may be used. For example, for the previously mentioned three states: a couched state, an upright state and a walking state, waking requires the most physical force and hence is considered to be a more unlikely state to occur. Thus, a threshold condition for being selected as the final representative value Bᵢ may be provided for each component of a vector. For example, if the user has ambulated for 6 seconds or more, the walking state may be preferentially selected as the intermediate representative value A_{j,i}.

In this case, the intermediate representative value A_{j,i} is represented by Formula (6):

Formula 6

A_{j,i}=j if N_{2,i}≥6 (6)

In this case, if (N_{0,i}, N_{1,i}, N_{2,i})=(30, 20, 10), then A_{j,i}=2, providing a different result from that of the Formula (4) described above. Alternatively, if there is no ambulation of the user for 6 seconds or more, a state (0 or 1) with a longer period of either the couched period N_{0,i} or the upright period N_{1,i} may be determined by majority decision as the intermediate representative value A_{j,i} or one with a greater value of j may be preferentially adopted.

When the final representative value Bᵢ is calculated based on the intermediate representative value A_{j,i} calculated by means of Formula (6) or a given conditional statement, including use of a threshold, the value of the final representative value Bᵢ may be similarly determined with setting of a condition. For example, an operation to determine the final representative value Bᵢ can be performed according to determination of considering that the user ambulated if there are one or more intermediate representative values A_{j,i} indicating the state of the user's ambulation among multiple intermediate representative values A_{j,i} to be used for the calculation of the final representative value Bᵢ.

However, as numerical values representing states such as whether the user walked or not are very abrupt in variations as compared to variations in the value of heart rate, blood pressure and the like, it is also possible to use the intermediate representative value A_{j,i} directly as equivalence to the final representative value Bᵢ. Particularly in the case of using multiple sensors in combination, the final representative value Bᵢ for a quantitative variable can agree with the user's intuition when it is calculated via an operation based on the intermediate representative value A_{j,i}, whereas for a qualitative variable, using the intermediate representative value A_{j,i} directly as the final representative value Bᵢ can agree with the user's intuition.

### Operational Sequence of Biological Information Analysis System

Next, operations in a case where the functions of the biological information analysis apparatus 1 according to this embodiment are implemented by a biological information analysis system including the sensor terminal 200, the relay terminal 300 and the external terminal 400 described in FIG. 6 are described with reference to the sequence diagram shown in FIG. 17. The respective functional blocks of the sensor terminal 200, the relay terminal 300, and the external terminal 400 are similar to the configurations described in FIG. 6.

Initially, the sensor terminal 200 is attached to the user 500 and measures posture and ambulation, for example, as the biological information of the user 500 (step S500). More specifically, the sensor terminal 200 detects acceleration data for the user 500 with a 3-axis acceleration sensor (the sensor 201). The sensor data acquisition unit 202 acquires acceleration data from the sensor 201 and measures the couched state, the upright state, and the walking state of the user 500 from inclination and/or body motion based on the acceleration data. Time-series data for the measured biological information indicating the posture and ambulation states of the user is stored in the data storage unit 203.

Next, the sensor terminal 200 transmits the measured biological information indicating the posture and ambulation of the user to the relay terminal 300 over the communication network NW (step S501). More specifically, the data transmission unit 204 reads the time-series data for the biological information indicating the state of the user from the data storage unit 203 and transmits it to the relay terminal 300 over the communication network NW.

After the relay terminal 300 receives the time-series data for the biological information indicating the state of the user 500 from the sensor terminal 200, the first calculation unit 305 calculates intermediate representative values with the time-series data for the state of the user 500 at intervals of a set period, e.g., at intervals of 60 seconds (step S502). More particularly, the first calculation unit 305 calculates as an intermediate representative value the proportions of the periods during which the state of the user 500 is couched, upright, and walking at intervals of 60 seconds using the Formula (4) mentioned above, for example. The calculated intermediate representative value is stored in the data storage unit 302.

Subsequently, the second calculation unit 306 calculates the final representative value based on the calculated intermediate representative value (step S503). More particularly, the second calculation unit 306 may use the Formula (5) above to calculate the final representative value by means of the mode. Next, the calculated final representative value is transmitted from the relay terminal 300 to the external terminal 400 (step S504).

Subsequently, the external terminal 400 receives the final representative value. The external terminal 400 performs presentation processing based on the final representative value (step S505), displaying the final representative value on the display device or generating and outputting assistance information for the user 500.

As described above, the biological information analysis apparatus 1 according to the fifth embodiment also can be applied to a sensor that measures biological information equivalent to a qualitative variable because it uses proportions of certain periods as intermediate representative values of biological information.

### Sixth Embodiment

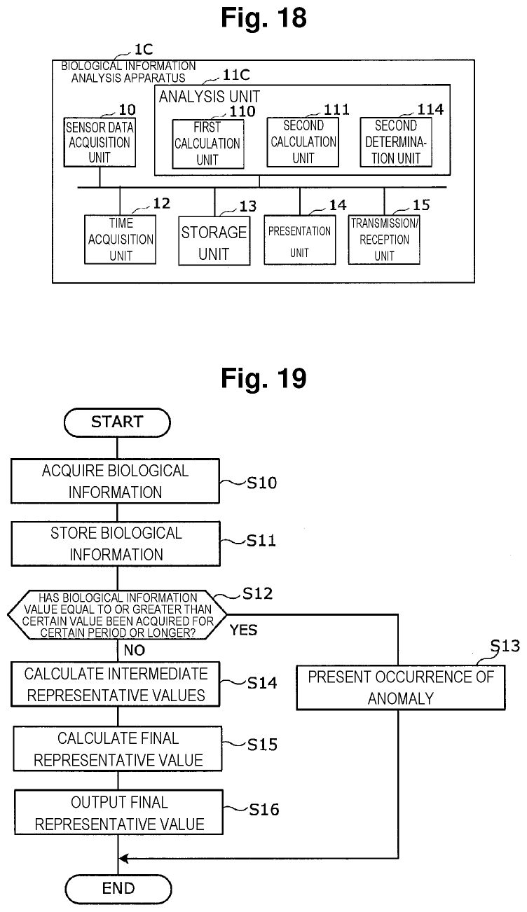

Next, a sixth embodiment of the present invention is described. A biological information analysis apparatus 1C according to the sixth embodiment includes in an analysis unit 11C a second determination unit 114 that determines that an anomaly has occurred when the value of sensor data falls in a set range of values for a certain period, as shown in FIG. 18. The other components of the biological information analysis apparatus 1C are similar to the first embodiment.

In the following, the operation of the biological information analysis apparatus 1C according to this embodiment is described with the flowchart of FIG. 19. First, the following processing is performed with the sensor 105 worn by the user.

The sensor data acquisition unit 10 acquires biological information measured by the sensor 105 worn by the user (step S10). More particularly, the sensor data acquisition unit 10 acquires the biological information and outputs time-series data in which the biological information and times of measurement are associated with each other. Next, the time-series data for biological information is stored in the storage unit 13 (step S11).

Then, if the value of the acquired time-series data for biological information is equal to or greater than a certain set value for a certain period (step S12: YES), for example, the second determination unit 114 determines that an anomaly is occurring in the biological information and causes the presentation unit 14 to present information indicating the occurrence of the anomaly (step S13). In this case, it is possible that an emergent situation is occurring with the user wearing the sensor 105, so that the processing is ended without calculating representative values for the biological information.

Biological information sometimes contains information for which emergent handling is desirable. However, as intermediate representative values and the final representative value are summary values, there is fear that information indicating an occurrence of an anomaly, such as an emergency with the user, can be dropped. In this embodiment, when a particular behavior with suspected emergency occurs in the sensor data acquired by the sensor data acquisition unit 10, information indicating an occurrence of an anomaly is output without going through the calculation of intermediate representative values and the final representative value. For example, by presenting the information indicating an occurrence of an anomaly on the presentation unit 14 or transmitting it to outside from the transmission/reception unit 15, the occurrence of an emergent situation with the user can be appropriately reported.

The second determination unit 114 determines that an anomaly has occurred when the value of the biological information acquired by the sensor data acquisition unit 10 is equal to or greater than a certain value or equal to or smaller than the value for a certain period, for example. Specifically, the second determination unit 114 determines that an anomaly has occurred when the heart rate is equal to or greater than 180 bpm or it is equal to or smaller than 40 bpm for 10 seconds, for example. A case of the heart rate being equal to or smaller than 40 bpm or equal to or greater than 180 bpm is rare except where the user has tachycardia or the user is a robust athlete; in many cases, it is due to measurement anomalies of the sensor 105.

The second determination unit 114 monitors for appearance of a value which is not realistic as a value in the range that can be assumed by normal biological information, and if such a value continues for a certain period, it determines that an anomaly has occurred, that is, regards it as an occurrence of an emergency, and transmits a signal to the presentation unit 14 and/or the transmission/reception unit 15. This can provide an opportunity to check the measurement state of the sensor 105 early and the second determination unit 114 can return the sensor 105 to a correct measurement state.

In addition to the previously described criterion for determination, the second determination unit 114 may also determine that an anomaly has occurred when a sensor data value within a certain range has been acquired for a certain period, for example. Specifically, the second determination unit 114 uses the time acquired via the time acquisition unit 12 to determine that an anomaly has occurred when the state of the user being couched (o) at 12 p.m. continues for a certain period. For example, in an inpatients' ward, schedules are strictly set such as 12 p.m. being a meal time. Thus, if the user is couched during this time slot, cases such as the user being left unattended by mistake or the sensor 105 being incorrectly attached are suspected. The second determination unit 114 determines that an anomaly has occurred based on such a biological information value and informs about it, thereby providing an opportunity to check the user's state and the measurement state of the sensor 105 early and allowing the sensor 105 to return to a correct measurement state.

Meanwhile, when it is determined that no anomaly has occurred at step S12 (step S12: NO), the first calculation unit 110 calculates intermediate representative values for the time-series data for the biological information measured at step S10 (step S14). Subsequently, after a predetermined amount of time has passed, the second calculation unit 111 calculates the final representative value of the time-series data for biological information based on the intermediate representative values calculated at step S14 (step S15). Then, the analysis unit 11C outputs the final representative value calculated at step S15 (step S16).

The second determination unit 114 may determine whether an anomaly has occurred or not based on the intermediate representative values calculated by the first calculation unit 110 or the final representative value calculated by the second calculation unit 111. Specifically, consider a case where the sensor 105 is a position sensor, e.g., GPS. In such a case, if the position coordinates of the user stay in a certain range (e.g., within a radius of 5 m) for a certain period (e.g., about one hour), there is a concern like the user being drowned if the estimated position is a bathroom, for example. Thus, it is desirable that the second determination unit 114 determine that an anomaly has occurred and provide a notification.

Alternatively, the second determination unit 114 may determine that an anomaly has occurred and provide a notification when position information of the user fails to be confirmed at a particular time within a certain range. For example, if the user does not coincide with the position coordinates of a sick room about 30 minutes after 21 o'clock, which is the lights-out time in a hospital, there is a concern like the user is going out alone or the like.

As described above, the sixth embodiment determines whether an anomaly has occurred or not based on the value of the acquired biological information of the user, thus providing an opportunity to check the user state or the measurement state of the sensor 105 early and allowing the sensor 105 to return to a correct measurement state.

### Seventh Embodiment

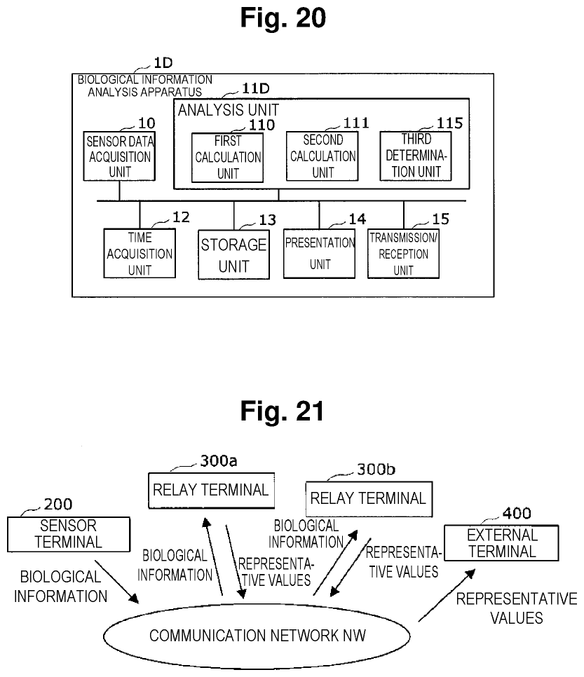

Next, a seventh embodiment of the present invention is described. A biological information analysis apparatus 1D according to the seventh embodiment includes in an analysis unit 11D a third determination unit 115 that determines whether an emergent situation with the user has occurred or not based on status of communication with the sensor terminal 200, as shown in FIG. 20. The other components of the biological information analysis apparatus 1D are similar to the first embodiment.

An example of a biological information analysis system for implementing the biological information analysis apparatus 1D according to this embodiment is shown in FIG. 21. The sensor terminal 200, multiple relay terminals 300a, 300b, and the external terminal 400 are connected with each other via the communication network NW. The analysis unit 11D with the third determination unit 115 is provided in each of the relay terminals 300a, 300b, for example. The configurations of the sensor terminal 200, the relay terminals 300a, 300b, and the external terminal 400 are similar to those in the first embodiment.

The relay terminals 300a, 300b are able to perform communication with the sensor terminal 200 within a predetermined range from the positions where they are installed. The sensor terminal 200 and the relay terminals 300a, 300b can be uniquely identified by a MAC address or an IP address. If where in the user's living space a terminal with particular MAC address and IP address is installed is known in advance, the location of the user present indoors can be determined. For example, with GPS, estimation accuracy of an indoor position is significantly decreased, whereas position estimation with the MAC address or IP address of a terminal can provide reliable position estimation even indoors.

The third determination unit 115 monitors such identification information unique to terminals, and determines that an emergent situation with the user has occurred when the relay terminal 300a, 300b is performing communication with a particular sensor terminal 200 for a certain period or longer. Information indicating the occurrence of an emergency as determined by the third determination unit 115 can be output to the outside via the presentation unit 14 and/or the transmission/reception unit 15.

For example, installing the relay terminals 300a, 300b at desired locations within a building respectively enables determination of the location of the user wearing the sensor terminal 200 within the building. In this case, the situation is such that the sensor terminal 200 having a particular MAC address transmits biological information to the relay terminal 300a or 300b having a particular IP address. Thus, the user and the current location of the user can be identified from the combination of those addresses.

As a specific example, installing the relay terminals 300a, 300b at places that are less observable by people other than the user (such as a washroom and a bathroom) allows the location and action of the user to be ascertained from staying of the user wearing the sensor terminal 200 in those places. The third determination unit 115 determines some emergent situation has occurred with the user wearing the sensor terminal 200 if the identification information of the sensor terminal 200 with which the relay terminal 300a, 300b is communicating remains the same and unchanged for a certain period, and provides a notification. In particular, the user continuing to stay in a place less observable by people other than the user for a long time can imply that some abnormality is occurring in the user's condition. In such a situation, an opportunity to check the user's safety early can be provided.

Alternatively, it may be determined that some emergent situation has occurred with the user when the identification information of the sensor terminal 200 has failed to be acquired for a certain period at the relay terminal 300a, 300b. For example, if the user wearing the sensor terminal 200 goes outside the area of the relay terminal 300a, the MAC address of the sensor terminal 200 will cease being acquired at the relay terminal 300a. For example, for a user with dementia, if the MAC address of the sensor terminal 200 has ceased being acquired after it was last recognized by a relay terminal 300a installed at an entrance, the user can possibly be going out alone and getting lost. In such a situation, an opportunity to discover an emergency with the user early can be provided.

The third determination unit 115 is not limited to being provided in the relay terminals 300a, 300b but the external terminal 400 may include the third determination unit 115.

While the biological information analysis apparatus, the biological information analysis method, and the biological information analysis system according to embodiments of the present invention have been described, the present invention is not limited to the described embodiments and various modifications conceivable by those skilled in the art may be made within the scope of the invention as set forth in the claims.

Although the embodiments were described for the case of using heart rate, acceleration, posture, or ambulation as biological information that is measured by the sensor data acquisition unit 10, biological information is not limited to them but may also be myogenic potential, heartbeat, pulse, blood pressure, respiration, speed of travel, location, action, exercise intensity, body motion, active mass etc., for example.

### REFERENCE SIGNS LIST

1 biological information analysis apparatus
10, 202 sensor data acquisition unit
11, 304 analysis unit
12, 303 time acquisition unit
13 storage unit
14, 404 presentation unit
15 transmission/reception unit
110 first calculation unit
11 second calculation unit
101 bus
102 CPU
103 main storage
104 communication interface
105, 201 sensor
106 auxiliary storage
107 clock
108 input/output device
109 display device
200 sensor terminal
300 relay terminal
400 external terminal
203, 302, 402 data storage unit
204, 307 data transmission unit
301, 401 data reception unit
403 presentation processing unit.

1.-10. (canceled)

11. A biological information analysis apparatus comprising:
a sensor data acquirer configured to acquire biological information;
a storage configured to store time-series data for the biological information acquired by the sensor data acquirer; and
an analyzer configured to calculate statistical representative values of the time-series data for the biological information stored in the storage in a stepwise manner, wherein the analyzer includes:
a first calculator configured to calculate first representative values from the time-series data for the biological information stored in the storage at intervals of a set period; and
a second calculator configured to calculate a second representative value from at least one of the first representative values or a plurality of consecutive ones of the first representative values.

12. The biological information analysis apparatus according to claim 11, further comprising:
an adjuster configured to determine a number of the first representative values to be used by the second calculator to calculate the second representative value, based on the number of the first representative values that are already calculated,
wherein the second calculator is configured to calculate the second representative value based on the consecutive ones of the first representative values corresponding to the number of the first representative values to be used as determined by the adjuster.

13. The biological information analysis apparatus according to claim 11, further comprising:
a first determiner configured to monitor for lack in the time-series data for the biological information and determine whether or not the lack is a long-term lack with a period of occurrence equal to or greater than a threshold, at intervals of the set period,
wherein the first calculator is configured to not calculate the first representative values during the set period when the lack determined to be a long-term lack by the first determiner is occurring.

14. The biological information analysis apparatus according to claim 13, further comprising a second determiner configured to determine that an anomaly has occurred when a value of the time-series data for the biological information falls in a set range of values for a predetermined period.

15. A biological information analysis method comprising:
a sensor data acquisition step of acquiring biological information;
a storage step of storing time-series data for the biological information in a storage; and
an analysis step of calculating statistical representative values of the time-series data for the biological information stored in the storage in a stepwise manner, wherein the analysis step includes:
a first calculation step of calculating first representative values from the time-series data for the biological information at intervals of a set period; and
a second calculation step of calculating a second representative value from one of the first representative values or a plurality of consecutive ones of the first representative values.

16. The biological information analysis method according to claim 15, further comprising:
an adjustment step of determining a number of the first representative values to be used in the second calculation step to calculate the second representative value, based on the number of the first representative values that are already calculated,
wherein the second calculation step includes calculating the second representative value based on the consecutive ones of the first representative values corresponding to the number of the first representative values to be used as determined in the adjustment step.

17. The biological information analysis method according to claim 15, further comprising:
a first determination step for monitoring for a lack in the time-series data for the biological information and determining whether or not the lack is a long-term lack with a period of occurrence equal to or greater than a threshold, at intervals of the set period,
wherein the first calculation step does not include calculating the first representative values during the set period when the lack determined to be a long-term lack in the first determination step is occurring.

18. The biological information analysis method according to claim 17, further comprising a second determination step for determining that an anomaly has occurred when a value of the time-series data for the biological information falls in a set range of values for a predetermined period.

19. A biological information analysis system comprising:
a sensor terminal configured to output biological information;
a relay terminal configured to receive the biological information output by the sensor terminal and output the biological information received from the relay terminal;
an external terminal configured to receive the biological information output by the sensor terminal or by the relay terminal and cause the biological information received from the sensor terminal or the relay terminal to be displayed on a display device;
a storage configured to store time-series data for the biological information; and
an analyzer configured to calculate statistical representative values of the time-series data for the biological information stored in the storage in a stepwise manner, wherein the analyzer includes:
a first calculator configured to calculate first representative values from the time-series data for the biological information stored in the storage at intervals of a set period; and
a second calculator configured to calculate a second representative value from one of the first representative values or a plurality of consecutive ones of the first representative values.

20. The biological information analysis system according to claim 19, wherein:
the sensor terminal comprises a first analyzer of the analyzer;
the relay terminal comprises a second analyzer of the analyzer; and
the external terminal comprises a third analyzer of the analyzer.

21. The biological information analysis system according to claim 20, wherein:
the first analyzer or the second analyzer is the first calculator; and
the third analyzer is the second calculator.

22. The biological information analysis system according to claim 19, wherein:
the relay terminal is at a preset position and is capable of communication within a predetermined range from the preset position; and
the relay terminal comprises a determiner configured to determine that an anomaly has occurred when communication has been established between the relay terminal and the sensor terminal for a predetermined period.

23. The biological information analysis system according to claim 19, wherein:
the relay terminal is at a preset position and is capable of communication within a predetermined range from the preset position; and
the external terminal comprises a determiner configured to determine that an anomaly has occurred when communication has been established between the relay terminal and the sensor terminal for a predetermined period.

24. The biological information analysis system according to claim 19, wherein each of the first representative values and the second representative value is an average of the biological information for a respective predetermined period.

25. The biological information analysis system according to claim 19, wherein each of the first representative values and the second representative value is a proportion of the biological information for a respective predetermined period.
